(19) United States
(12) Patent Application Publication
(10) Pub. No.: US 2023/0113585 A1
(43) Pub. Date: Apr. 13, 2023

(54) METHOD AND COMPUTING SYSTEM FOR ESTIMATING BINDING FREE ENERGY OF MUTANT PROTEIN COMPLEX

(71) Applicant: GRAPHEN, INC., New York, NY (US)

(72) Inventors: Yi-Ting CHEN, Taipei City (TW); Sing-Han HUANG, Taipei City (TW); Ching-Yung LIN, Scarsdale, NY (US); Xiang-Yu LIN, Taipei City (TW); Cheng-Tang WANG, Taipei City (TW)

(21) Appl. No.: 17/865,140

(22) Filed: Jul. 14, 2022

Related U.S. Application Data
(60) Provisional application No. 63/248,804, filed on Sep. 27, 2021.

(51) Int. Cl. G16B 15/30; G16B 40/00

(52) U.S. Cl. CPC G16B 15/30; G16B 40/00

(57) ABSTRACT

A method includes steps of: based on protein structure data, selecting a residue pair that includes a specific residue and a paired residue respectively of two wild-type protein chains of a protein complex; determining a mutant residue to substitute for the specific residue; for a target interface between the mutant residue and the paired residue, calculating an atomic distance and an atomic interaction force based on the protein structure data and amino acid structure data; and estimating binding free energy of the target interface by feeding the atomic distance, the atomic interaction force, and physicochemical information related to the specific residue and the mutant residue into a deep neural network.

### CROSS-REFERENCE TO RELATED APPLICATION

This application claims priority of U.S. Provisional Pat. Application No. 63/248804, filed on Sep. 27, 2021.

### FIELD

The disclosure relates to a method and a computing system for estimating binding free energy of a mutant protein complex.

### BACKGROUND

FIG. 1 illustrates an interaction between a receptor-binding domain (RBD) of a spike protein (also known as an S protein) of a severe acute respiratory syndrome coronavirus 2 (SARS-CoV-2) and angiotensin-converting enzyme 2 (ACE2). Because of N501Y mutation of S protein in which an asparagine residue in a wild-type S protein (N501, shown in the left part of FIG. 1) is substituted by a tyrosine residue (Y501, shown in the right part of FIG. 1), an atomic interaction (denoted by dashed lines) is additionally formed between the tyrosine residue (Y501) and an aspartic acid residue (D) of the ACE2 besides the atomic interactions formed between the tyrosine residues (Y501) with a lysine residue (K) and a tyrosine reside (Y) of the ACE2. Compared with the asparagine residue (N501) in the wild-type S protein, the tyrosine residue (Y501) in the mutant S protein is more adjacent to the lysine residue (K) and the tyrosine reside (Y) of the ACE2. Thus, binding of mutant S protein to ACE2 is strengthened, making SARS-CoV-2 relatively more infectious to humans.

Conventionally, a wet-lab approach is adopted to study protein-protein interaction in a mutant protein complex. For example, a mutagenesis technique is utilized to change a specific amino acid residue of a wild-type protein complex to a mutant amino acid residue, thereby obtaining a mutant protein complex. Moreover, an isothermal titration calorimetry (ITC) technique is utilized to determine thermodynamic parameters of protein-protein interaction of the mutant protein complex, so as to determine the effect of amino acid mutations on protein-protein interaction. However, such approach requires extreme precautions for laboratory safety and extensive expertise, and is costly, labor intensive and time-consuming.

### SUMMARY

Therefore, an object of the disclosure is to provide a method and a computing system for estimating binding free energy of a mutant protein complex that can alleviate at least one of the drawbacks of the prior art.

According to one aspect of the disclosure, the method is to be implemented by a computing system. The method includes steps of:
from protein structure data containing spatial coordinate sets respectively of all atoms of a reference protein complex, obtaining spatial coordinate sets respectively of all heavy atoms of the reference protein complex, the reference protein complex including two wild-type protein chains;
for every two heavy atoms that belong respectively to the wild-type protein chains of the reference protein complex, calculating an Euclidean distance between the two heavy atoms as an interatomic distance based on the spatial coordinate sets respectively of the two heavy atoms;
identifying, based on the interatomic distances calculated in the step of calculating an Euclidean distance, all interaction interfaces in the reference protein complex, wherein each of the interaction interfaces is between two residues respectively of the wild-type protein chains and wherein a distance between two α-carbons respectively of the residues is less than 5 Å;
selecting one of the interaction interfaces that is related to a specific residue pair, the specific residue pair including a specific residue at a site of interest in one of the wild-type protein chains of the reference protein complex and a paired residue in the other one of the wild-type protein chains of the reference protein complex;
determining, according to information related to properties of side-chain dihedral angles and bond rotation of amino acids, a mutant residue that possibly results from mutation of the specific residue of the reference protein complex and that changes the reference protein complex into a mutant protein complex;
obtaining an inferred rotation angle that is related to a side chain of the specific residue of the reference protein complex from amino acid structure data, the amino acid structure data containing information related to properties of backbone dihedral angles, side-chain dihedral angles and bond rotation of amino acids;
calculating spatial coordinate sets respectively of all heavy atoms of the mutant residue based on the spatial coordinate sets of all heavy atoms of the specific residue of the reference protein complex and the inferred rotation angle;
for a target interface between the mutant residue and a paired residue of the mutant protein complex that respectively correspond to the specific residue and the paired residue of the specific residue pair of the reference protein complex,
   for every two heavy atoms respectively of the mutant residue and the paired residue of the mutant protein complex, calculating a value of atomic-level energy and an Euclidean distance based on the spatial coordinate sets of the heavy atoms of the reference protein complex and the spatial coordinate sets of the heavy atoms of the mutant residue of the mutant protein complex, and
   calculating, based on the values of atomic-level energy and the Euclidean distances thus calculated, an atomic distance related to the target interface and an atomic interaction of the target interface;
obtaining relevant information that is related to the specific residue of the reference protein complex and the mutant residue of the mutant protein complex from amino acid physicochemical properties data, the amino acid physicochemical properties data containing information related to physicochemical properties of amino acids; and
estimating binding free energy of the target interface by feeding, into a model for estimating binding free energy, the atomic distance related to the target interface, the atomic interaction of the target interface and the relevant information, wherein the model for estimating binding free energy is implemented by a deep neural network (DNN).

According to another aspect of the disclosure, the computing system includes a storage device, an input module, an output module and a processor.

The storage device is configured to store amino acid structure data, amino acid physicochemical properties data and a model for estimating binding free energy. The amino acid structure data contains information related to properties of backbone dihedral angles, side-chain dihedral angles and bond rotation of amino acids. The amino acid physicochemical properties data contains information related to physicochemical properties of amino acids. The model for estimating binding free energy is implemented by a deep neural network.

The input module is configured to receive protein structure data that contains spatial coordinate sets of all atoms of a reference protein complex. The reference protein complex includes two wild-type protein chains.

The processor is electrically connected to the storage device, the input module and the output module. The processor is configured to obtain spatial coordinate sets respectively of all heavy atoms of the reference protein complex from the protein structure data. The processor is further configured to, for every two heavy atoms that belong respectively to the wild-type protein chains of the reference protein complex, calculate an Euclidean distance between the two heavy atoms as an interatomic distance based on the spatial coordinate sets respectively of the two heavy atoms. The processor is further configured to identify, based on the interatomic distances thus calculated, all interaction interfaces in the reference protein complex, wherein each of the interaction interfaces is between two residues respectively of the wild-type protein chains and wherein a distance between two α-carbons respectively of the residues is less than 5 Å. The processor is further configured to select one of the interaction interfaces that is related to a specific residue pair. The specific residue pair includes a specific residue at a site of interest in one of the wild-type protein chains of the reference protein complex and a paired residue in the other one of the wild-type protein chains of the reference protein complex. The processor is further configured to determine, according to information related to properties of side-chain dihedral angles and bond rotation of amino acids, a mutant residue that possibly results from mutation of the specific residue of the reference protein complex and that changes the reference protein complex into a mutant protein complex. The processor is further configured to obtain an inferred rotation angle that is related to a side chain of the specific residue of the reference protein complex from the amino acid structure data. The processor is further configured to calculate spatial coordinate sets respectively of all heavy atoms of the mutant residue based on the spatial coordinate sets of all heavy atoms of the specific residue of the reference protein complex and the inferred rotation angle. For a target interface between the mutant residue and a paired residue of the mutant protein complex that respectively correspond to the specific residue and the paired residue of the specific residue pair of the reference protein complex, the processor is further configured to, for every two heavy atoms respectively of the mutant residue and the paired residue of the mutant protein complex, calculate a value of atomic-level energy and an Euclidean distance based on the spatial coordinate sets of the heavy atoms of the reference protein complex and the spatial coordinate sets of the heavy atoms of the mutant residue of the mutant protein complex, and calculate, based on the values of atomic-level energy and the Euclidean distances thus calculated, an atomic distance related to the target interface and an atomic interaction of the target interface. The processor is further configured to obtain relevant information that is related to the specific residue of the reference protein complex and the mutant residue of the mutant protein complex from the amino acid physicochemical properties data. The processor is further configured to estimate binding free energy of the target interface by feeding, into the model for estimating binding free energy, the atomic distance related to the target interface, the atomic interaction of the target interface and the relevant information. The processor is further configured to control the output module to present the binding free energy of the target interface thus estimated.

### BRIEF DESCRIPTION OF THE DRAWINGS

Other features and advantages of the disclosure will become apparent in the following detailed description of the embodiment with reference to the accompanying drawings, of which:

FIG. 1 is a schematic diagram illustrating interaction between angiotensin-converting enzyme 2 (ACE2) and a receptor-binding domain of a spike protein;

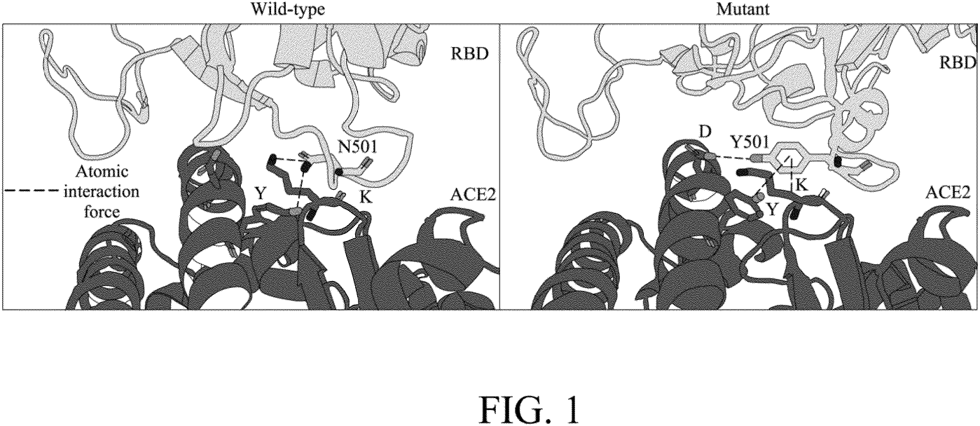

FIG. 2 is a block diagram illustrating an example of a computing system for estimating binding free energy of a mutant protein complex according to an embodiment of the disclosure;

FIG. 3 is a schematic diagram illustrating an example of a model for estimating binding free energy according to an embodiment of the disclosure;

FIG. 4 is a schematic diagram illustrating a result of validating performance of the model for estimating binding free energy;

FIG. 5 is a schematic diagram illustrating an amino acid structure;

FIG. 6 is a flow chart illustrating a method for estimating binding free energy of a mutant protein complex according to an embodiment of the disclosure; and

FIG. 7 is a schematic diagram illustrating an interaction interface between two protein chains.

### DETAILED DESCRIPTION

Before the disclosure is described in greater detail, it should be noted that where considered appropriate, reference numerals or terminal portions of reference numerals have been repeated among the figures to indicate corresponding or analogous elements, which may optionally have similar characteristics.

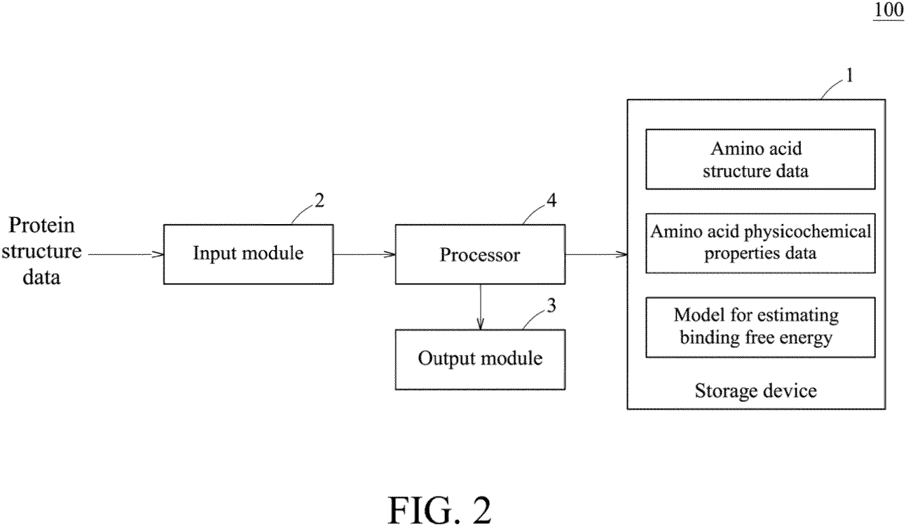

Referring to FIG. 2, an embodiment of a computing system 100 for estimating binding free energy of a mutant protein complex according to the disclosure is illustrated. The computing system 100 may be implemented to be a desktop computer, a laptop computer, a notebook computer or a tablet computer, but implementation thereof is not limited to what are disclosed herein and may vary in other embodiments.

The computing system 100 includes a storage device 1, an input module 2, an output module 3 and a processor 4. The processor 4 is electrically connected to the storage device 1, the input module 2 and the output module 3.

The storage device 1 may be implemented by random access memory (RAM), double data rate synchronous dynamic random access memory (DDR SDRAM), read only memory (ROM), programmable ROM (PROM), flash memory, a hard disk drive (HDD), a solid state disk (SSD), electrically-erasable programmable read-only memory (EEPROM) or any other volatile/non-volatile memory devices, but is not limited thereto. The storage device 1 is configured to store amino acid structure data, amino acid physicochemical properties data and a model for estimating binding free energy.

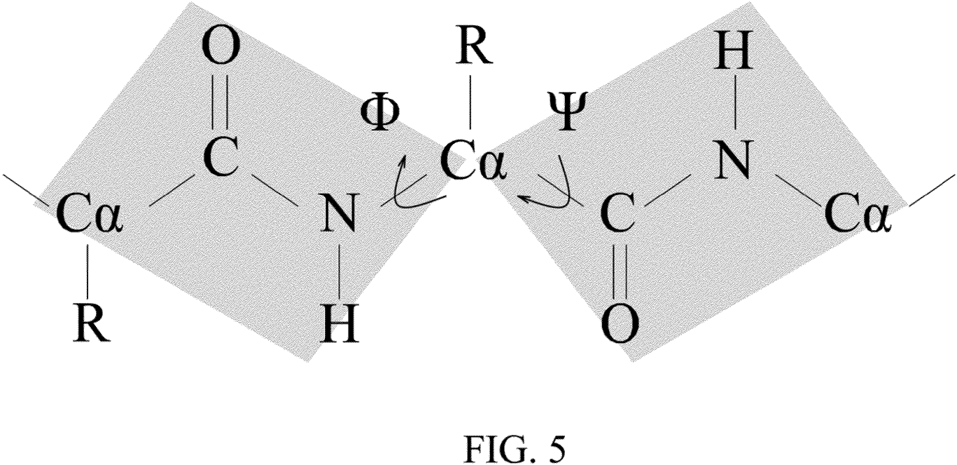

The amino acid structure data reveals information related to properties of backbone dihedral angles, side-chain dihedral angles and bond rotation of amino acids. It is worth to note that in regard to amino acids of a protein chain (see FIG. 5) , two bonds “Cα - N” and “Cα - C” that are respectively at two sides of an α-carbons (Cα) are each freely rotatable. In addition, chains “Cα - C - N - Cα” at two sides of the α-carbons (Cα) respectively define two planes (which are colored in grey in FIG. 5). An internal angle between two intersecting planes defined by chain “C - N - Cα - C” is referred to as a backbone dihedral angle “Φ”, an internal angle between two intersecting planes defined by chain “N - Cα - C -N” is referred to as a backbone dihedral angle “Ψ”, and an internal angle between two intersecting planes defined by chain “N - Cα - Cβ - XG” (not shown) is referred to as a sidechain dihedral angle “Xₙ” (where n is an integer such as one). Since properties of backbone dihedral angles, side-chain dihedral angles and bond rotation of amino acids have been well known to one skilled in the relevant art, detailed explanation of the same is omitted herein for the sake of brevity.

The amino acid physicochemical properties data contains information that is related to physicochemical properties of at least 21 amino acids, including alanine (i.e., Ala or A), arginine (i.e., Arg, R), asparagine (i.e., Asn or N), aspartate (i.e., Asp or D), cysteine (i.e., Cys or C), glutamine (i.e., Gln or Q) , glutamate (i.e., Glu or E), glycine (i.e., Gly or G), histidine (i.e., His or H), isoleucine (i.e., Ile or I), leucine (i.e., Leu or L), lysine (i.e., Lys or K), methionine (i.e., Met or M), phenylalanine (i.e., Phe or F), proline (i.e., Pro or P), serine (i.e., Ser or S), threonine (i.e., Thr or T), tryptophan (i.e., Trp or W) , tyrosine (i.e., Tyr or Y), valine (i.e., Val or V), and selenocysteine (i.e., Sec or U), but are not limited to what are disclosed herein. According to physicochemical properties of side chains of amino acids, the amino acids can be exemplarily classified into amino acids with positively or negatively charged side chains, amino acids with polar side chains, amino acids with hydrophobic side chains, and amino acids with special side chains. Physicochemical properties of amino acids can be exemplarily encoded by five bits of binary digits, wherein for the five bits from left to right, a first bit being “1” indicates an amino acid with a positively charged side chain, a second bit being “1” indicates an amino acid with a negatively charged side chain, a third bit being “1” indicates an amino acid with a polar side chain, a fourth bit being “1” indicates an amino acid with a hydrophobic side chain, and a fifth bit being “1” indicates an amino acid with a special side chain. For example, physicochemical properties of asparagine (N), which is an amino acid with a polar side chain, would be encoded by binary digits “00100”. Since physicochemical properties of amino acids have been well known to one skilled in the relevant art, detailed explanation of the same is omitted herein for the sake of brevity.

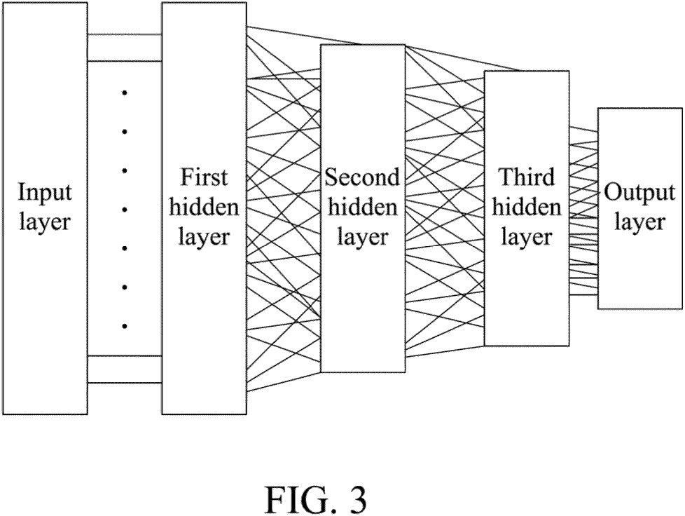

The model for estimating binding free energy is implemented by a deep neural network (DNN). Referring to FIG. 3, in this embodiment, the model for estimating binding free energy includes an input layer, three hidden layers and an output layer. For example, a first one of the three hidden layers (also referred to as a first hidden layer) includes 64 neurons and is implemented by a rectified linear unit (ReLU) activation function, a second one of the three hidden layers (also referred to as a second hidden layer) includes 32 neurons and is also implemented by the ReLU activation function, and a third one of the three hidden layers (also referred to as a third hidden layer) includes 16 neurons and is also implemented by the ReLU activation function.

In one embodiment, the input module 2 is embodied using a network interface controller or a wireless transceiver that supports wireless communication standards, such as Bluetooth®) technology standards, Wi-Fi technology standards and/or cellular network technology standards. The input module 2 is connected to a telecommunications network (not shown) for receiving data transmitted by a remote device (e.g., a data server).

In one embodiment, the input module 2 is embodied using a keyboard, a mouse, or a touch panel that is configured to present a graphical user interface. However, it should be noted that implementations of the input module 2 are not limited to what are disclosed herein and may vary in other embodiments.

The input module 2 is configured to receive protein structure data that contains spatial coordinate sets respectively of all atoms of a reference protein complex which includes two wild-type protein chains. The spatial coordinate sets may be represented by a 3-tuple in a Cartesian coordinate system, but is not limited thereto.

The output module 3 may be embodied using a display device (e.g., a liquid-crystal display (LCD), a light-emitting diode (LED) display, a plasma display panel, a projection display or the like). However, implementation of the output module 3 is not limited to the disclosure herein and may vary in other embodiments.

The processor 4 may be implemented by a central processing unit (CPU), a microprocessor, a micro control unit (MCU), a system on a chip (SoC), or any circuit configurable/programmable in a software manner and/or hardware manner to implement functionalities discussed in this disclosure.

The processor 4 is configured to obtain, from the protein structure data, spatial coordinate sets respectively of all heavy atoms of the reference protein complex. A heavy atom is an atom other than hydrogen, such as oxygen, nitrogen or carbon. For every two heavy atoms that belong respectively to the wild-type protein chains of the reference protein complex, the processor 4 is configured to calculate an Euclidean distance between the two heavy atoms as an interatomic distance based on the spatial coordinate sets respectively of the two heavy atoms.

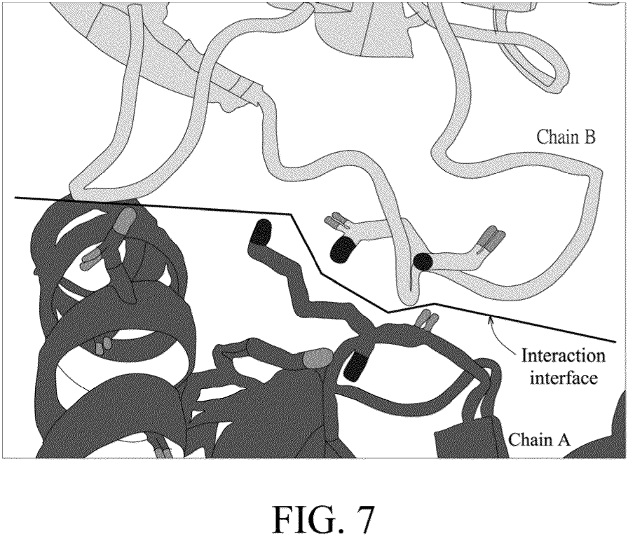

Subsequently, the processor 4 is configured to identify, based on the interatomic distances thus calculated, all interaction interfaces in the reference protein complex. Specifically, each of the interaction interfaces is between two residues respectively of the wild-type protein chains and a distance between two α-carbons (Cα) respectively of the residues is less than 5 Å. FIG. 7 illustrates an example of an interaction interface between two protein chains (i.e., “chain A” and “chain B”).

Then, the processor 4 is configured to select one of the interaction interfaces that is related to a specific residue pair. The specific residue pair includes a specific residue at a site of interest in one of the wild-type protein chains of the reference protein complex and a paired residue in the other one of the wild-type protein chains of the reference protein complex.

Thereafter, the processor 4 is configured to determine, according to information related to properties of side-chain dihedral angles and bond rotation of amino acids, a mutant residue that possibly results from mutation of the specific residue of the reference protein complex and that changes the reference protein complex into a mutant protein complex.

Additionally, the processor 4 is configured to obtain an inferred rotation angle that is related to a side chain of the specific residue of the reference protein complex from the amino acid structure data.

The processor 4 is further configured to calculate spatial coordinate sets respectively of all heavy atoms of the mutant residue based on the spatial coordinate sets of all heavy atoms of the specific residue of the reference protein complex and based on the inferred rotation angle.

For example, Table 1 below shows a lookup table which exemplifies information contained in the amino acid structure data, wherein a symbol “Φ” represents a backbone dihedral angle that is an internal angle between two intersecting planes defined by chain “C - N - Cα - C”, a symbol “Ψ” represents a backbone dihedral angle that is an internal angle between two intersecting planes defined by chain “N - Cα - C -N”, a symbol “X₁” represents a sidechain dihedral angle, and a symbol “ΔX₁” represents an inferred rotation angle. The inferred rotation angle “ΔX₁” can be determined based on the backbone dihedral angle “Φ”, the backbone dihedral angle “Ψ” and the sidechain dihedral angle “X₁”.

TABLE 1

| Φ | X₁ | ΔX₁ |
|---|---|---|
| 60° | -60° | -180° |
| -60° | 60° | 60° |
| 60° | -60° | 0° |
| -60° | 60° | -120° |
| Ψ | X₁ | ΔX₁ |
| 60° | -60° | -60° |
| -60° | 60° | 180° |
| 60° | -60° | 120° |
| -60° | 60° | 0° |

In a scenario of determining an inferred rotation angle that is related to a side chain of an asparagine residue 501 (i.e., N501) of a wild-type spike protein where a backbone dihedral angle “Φ” is -60° (i.e., Φ = -60°), a backbone dihedral angle “Ψ” is -60° (i.e., Ψ = -60°), and a sidechain dihedral angle “X₁” is 60° (i.e., X₁ = -60°), four inferred rotation angles ΔX₁ that are 60°, -120°, 180° and 0° (i.e., ΔX₁ = 60°, -120°, 180° and 0°) can be obtained by looking up Table 1 above. Afterwards, the processor 4 is capable of calculating spatial coordinate sets respectively of all heavy atoms of a tyrosine residue 501 (i.e., Y501), which results from mutation of the spike protein, based on the four inferred rotation angles ΔX₁ thus obtained and spatial coordinate sets of all heavy atoms of the asparagine residue 501 (N501). Specifically, for each of the four inferred rotation angles ΔX₁, a group of spatial coordinate sets respectively of all heavy atoms of the tyrosine residue 501 is obtained; that is to say, four groups of spatial coordinate sets of all heavy atoms of the tyrosine residue 501 are obtained and correspond respectively to the four inferred rotation angles ΔX₁. It is worth noting that an inferred rotation angle of 0 ° (i.e., ΔX₁ = 0 °) means that a side chain of the mutant residue would not be rotated with respect to that of the specific residue (i.e., the asparagine residue 501).

For a target interface between the mutant residue and a paired residue of the mutant protein complex that respectively correspond to the specific residue and the paired residue of the specific residue pair of the reference protein complex, the processor 4 is configured to implement the following calculations. The processor 4 calculates, for every two heavy atoms respectively of the mutant residue and the paired residue of the mutant protein complex (hereinafter referred to as “a mutant-residue-paired-residue heavy atom pair”), a value of atomic-level energy and an Euclidean distance based on the spatial coordinate sets of the heavy atoms of the reference protein complex and the spatial coordinate sets of the heavy atoms of the mutant residue of the mutant protein complex, and calculates, based on the values of atomic-level energy and the Euclidean distances thus calculated, an atomic distance (D) related to the target interface and an atomic interaction force (E) of the target interface.

Specifically, the processor 4 is configured to calculate, for each mutant-residue-paired-residue heavy atom pair of the mutant protein complex, the value of atomic-level energy as a sum of values of Van der Waals force, hydrogen bond, π-π stacking interaction and electrostatic force between the two heavy atoms of the pair. Thereafter, the processor 4 is further configured to calculate the atomic distance (D) as an average of the Euclidean distances of all mutant-residue-paired-residue heavy atom pairs of the mutant protein complex, and to calculate the atomic interaction force (E) as a sum of the values of atomic-level energy of all mutant-residue-paired-residue heavy atom pairs of the mutant protein complex.

Mathematically, the atomic distance (D) and the atomic interaction force (E) can be respectively expressed by

$$\text{D} = \frac{\sum {}_{i = 1}^{N} {d}_{i}}{N} , \text{and}$$

$$\text{E} = \sum {}_{i = 1}^{N} {e}_{i} ,$$

where N is a total number of the mutant-residue-paired-residue heavy atom pairs of the mutant protein complex, dᵢ represents an Euclidean distance of an i^{th} one of the mutant-residue-paired-residue heavy atom pairs of the mutant protein complex, and eᵢ represents an atomic-level energy of an i^{th} one of the mutant-residue-paired-residue heavy atom pairs of the mutant protein complex. Since calculations of Van der Waals force, hydrogen bond, π-π stacking interaction and electrostatic force have been well known to one skilled in the relevant art, detailed explanation of the same is omitted herein for the sake of brevity.

It should be noted that in a scenario where multiple inferred rotation angles are obtained and multiple groups of spatial coordinate sets of all heavy atoms of a mutant residue are thereby calculated, the processor 4 would eventually calculate, respectively for the multiple groups of spatial coordinate sets, multiple pairs of the atomic distance (D) and the atomic interaction force (E) (hereinafter also referred to as multiple candidates). Then, the processor 4 would reserve one of the multiple candidates, in which the atomic interaction force (E) is the smallest among the atomic interaction forces (E) of the candidates, for further processing.

Referring to the previous example where the four inferred rotation angles (ΔX₁ = 60°, -120°, 180° and 0°) are respectively used to calculate four groups of spatial coordinate sets of all heavy atoms of the tyrosine residue 501, the processor 4 would eventually calculate, respectively for the four groups of spatial coordinate sets, four pairs of the atomic distance and the atomic interaction force (D1, E1), (D2, E2), (D3, E3) and (D4, E4) that respectively correspond to the four inferred rotation angles (ΔX₁ = 60°, -120°, 180° and 0°). When the atomic interaction force (E4) is the smallest among the atomic interactions forces (El, E2, E3 and E4), the processor 4 would reserve the pair of the atomic distance and the atomic interaction force (D4, E4) for further processing.

The processor 4 is further configured to obtain relevant information that is related to the specific residue of the reference protein complex and the mutant residue of the mutant protein complex from the amino acid physicochemical properties data.

The processor 4 is further configured to estimate binding free energy of the target interface by feeding, into the model for estimating binding free energy, the atomic distance (D) related to the target interface, the atomic interaction force (E) of the target interface and the relevant information. The input layer of the model for estimating binding free energy is configured to receive the atomic distance (D), the atomic interaction force (E) and the relevant information, and the output layer of the model for estimating binding free energy is configured to output the binding free energy thus estimated.

It should be noted that the model for estimating binding free energy is trained in advance by using a plurality of training sets that respectively correspond to a plurality of training protein complexes. The training protein complexes are obtained by a computer over the Internet from a protein database such as “SKEMPI”, “AB-Bind”, “PROXIMATE” or “dbMPIKT”. Each of the training protein complexes includes at least one pair of training residues that are respectively in two protein chains of the training protein complex and that are related to a training interaction interface. Each of the training sets contains, for each of the at least one pair of training residues included in the corresponding one of the training protein complexes, an atomic distance that is related to the training interaction interface to which the pair of training residues are related, an atomic interaction force of the training interaction interface to which the pair of training residues are related, binding free energy of the training interaction interface to which the pair of training residues are related, and information related to physicochemical properties of amino acids that are related to the pair of training residues. After the model for estimating binding free energy has been trained by feeding the training sets thereinto, performance of the model for estimating binding free energy can be validated by using a plurality of validation sets, wherein contents of the validation sets are similar to those of the training sets.

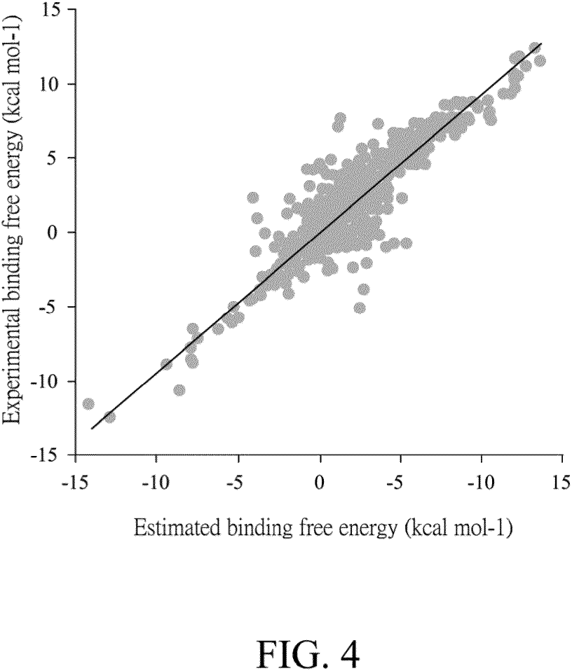

Referring to FIG. 4, a result of validating performance of the model for estimating binding free energy is illustrated. A vertical axis corresponds to an experimental binding free energy that is regarded as the ground truth, and a horizontal axis corresponds to an estimated binding free energy that is provided by the model for estimating binding free energy. Evidently, the model for estimating binding free energy can accurately estimate binding free energy of a protein complex, and a correlation between the experimental binding free energy and the estimated binding free energy shown in FIG. 4 is 0.91, which is better than a correlation of 0.74 calculated for an estimation made by using a BindProfX algorithm disclosed in the article entitled “BindProfX: Assessing Mutation-Induced Binding Affinity Change by Protein Interface Profiles with Pseudo-Counts” published by Peng Xiong, Chengxin Zhang, Wei Zheng and Yang Zhang in the Journal of Molecular Biology.

Finally, the processor 4 is configured to control the output module 3 to present the binding free energy of the target interface thus estimated. A person in the relevant art is able to analyze the mutant protein complex according to the binding free energy presented by the output module 3.

It should be noted that the lower the binding free energy, the stronger a binding force between two residues. Therefore, binding free energy of an interface between two residues respectively of two protein chains of a protein complex indicates how much binding force the two residues exert to bind the two protein chains together so as to stabilize the protein complex.

Moreover, when a specific residue of a wild-type protein complex is mutated and the wild-type protein complex becomes a mutant protein complex, binding free energy calculated for the mutant protein complex is helpful to determining how much impact the mutation has on functions of the wild-type protein complex.

With regards to drug design, for a predetermined interaction interface that is related to a protein of interest, a drug may be designed, with the assistance of the computing system 100 according to the disclosure, to favorably and exclusively bind to the protein of interest. In this way, efficiency and a success rate of drug development may be improved.

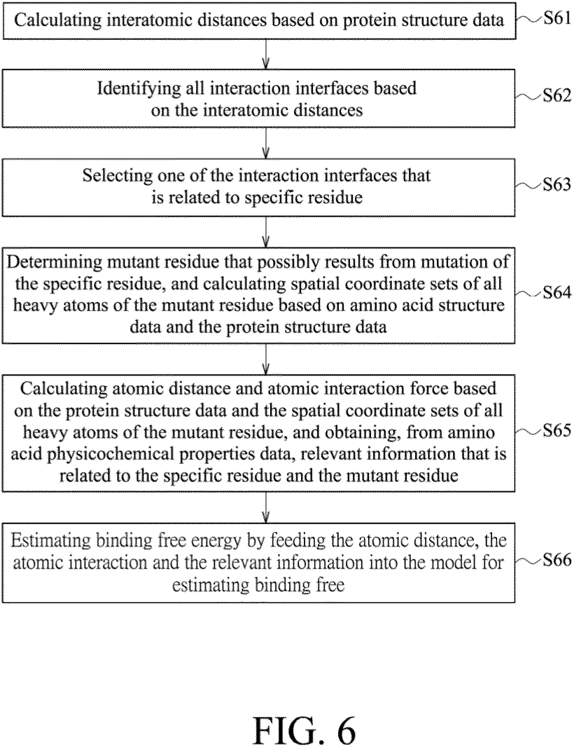

Referring to FIG. 6, an embodiment of a method for estimating binding free energy of a mutant protein complex according to the disclosure is illustrated. The method is to be implemented by the computing system 100 that is previously described. The method includes steps S61 to S66 delineated below.

In step S61, the processor 4 of the computing system 100 obtains, from the protein structure data, spatial coordinate sets respectively of all heavy atoms of the reference protein complex. For every two heavy atoms that belong respectively to the wild-type protein chains of the reference protein complex, the processor 4 calculates an Euclidean distance between the two heavy atoms as an interatomic distance based on the spatial coordinate sets respectively of the two heavy atoms.

In step S62, the processor 4 identifies, based on the interatomic distances calculated in S61, all interaction interfaces in the reference protein complex.

In step S63, the processor 4 selects one of the interaction interfaces that is related to a specific residue pair, wherein the specific residue pair includes a specific residue at a site of interest in one of the wild-type protein chains of the reference protein complex and a paired residue in the other one of the wild-type protein chains of the reference protein complex.

In step S64, the processor 4 determines, according to information related to properties of side-chain dihedral angles and bond rotation of amino acids, a mutant residue that possibly results from mutation of the specific residue of the reference protein complex and that changes the reference protein complex into a mutant protein complex. Subsequently, the processor 4 obtains, from the amino acid structure data, an inferred rotation angle that is related to a side chain of the specific residue of the reference protein complex, and calculates spatial coordinate sets respectively of all heavy atoms of the mutant residue based on the inferred rotation angle and the spatial coordinate sets of all heavy atoms of the specific residue of the reference protein complex.

In step S65, for a target interface between the mutant residue and a paired residue of the mutant protein complex that respectively correspond to the specific residue and the paired residue of the specific residue pair of the reference protein complex, the processor 4 calculates, for every two heavy atoms respectively of the mutant residue and the paired residue of the mutant protein complex (hereinafter referred to as “a mutant-residue-paired-residue heavy atom pair”), a value of atomic-level energy and an Euclidean distance based on the spatial coordinate sets of the heavy atoms of the reference protein complex and the spatial coordinate sets of the heavy atoms of the mutant residue of the mutant protein complex, and calculates, based on the values of atomic-level energy and the Euclidean distances thus calculated, an atomic distance (D) related to the target interface and an atomic interaction force (E) of the target interface.

In particular, the processor 4 calculates, for each mutant-residue-paired-residue heavy atom pair of the mutant protein complex, the value of atomic-level energy as a sum of values of Van der Waals force, hydrogen bond, π-π stacking interaction and electrostatic force between the two heavy atoms of the mutant-residue-paired-residue heavy atom pair. The processor 4 calculates the atomic distance (D) as an average of the Euclidean distances of all mutant-residue-paired-residue heavy atom pairs of the mutant protein complex. The processor 4 calculates the atomic interaction force (E) as a sum of the values of atomic-level energy of all mutant-residue-paired-residue heavy atom pairs of the mutant protein complex.

Further, the processor 4 obtains, from the amino acid physicochemical properties data, relevant information that is related to the specific residue of the reference protein complex and the mutant residue of the mutant protein complex.

In step S66, the processor 4 estimates binding free energy of the target interface by feeding, into the model for estimating binding free energy, the atomic distance (D) related to the target interface, the atomic interaction force (E) of the target interface and the relevant information.

To sum up, for the method and the computing system 100 according to the disclosure, a dry-lab approach is adopted to estimate binding free energy of a mutant protein complex. For a target interface between a mutant residue and a paired residue of a mutant protein complex, an atomic distance and an atomic interaction force are calculated based on the protein structure data that contains spatial coordinate sets respectively of all atoms of a reference protein complex, and on the amino acid structure data that contains information related to properties of backbone dihedral angles, side-chain dihedral angles and bond rotation of amino acids. Thereafter, the model for estimating binding free energy, which is implemented by a deep neural network, is utilized to estimate binding free energy of the target interface based on the atomic distance, the atomic interaction force, and relevant information that is related to physicochemical properties of the mutant residue of the mutant protein complex and a specific residue, which corresponds to mutant residue, of the reference protein complex. In this way, binding free energy of a mutant protein complex may be efficiently and accurately estimated without conducting biochemical experimentation.

In the description above, for the purposes of explanation, numerous specific details have been set forth in order to provide a thorough understanding of the embodiment. It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects, and that one or more features or specific details from one embodiment may be practiced together with one or more features or specific details from another embodiment, where appropriate, in the practice of the disclosure.

While the disclosure has been described in connection with what is considered the exemplary embodiment, it is understood that this disclosure is not limited to the disclosed embodiment but is intended to cover various arrangements included within the spirit and scope of the broadest interpretation so as to encompass all such modifications and equivalent arrangements.

What is claimed is:

1. A method for estimating binding free energy of a mutant protein complex to be implemented by a computing system, the method comprising steps of:
from protein structure data containing spatial coordinate sets respectively of all atoms of a reference protein complex, obtaining spatial coordinate sets respectively of all heavy atoms of the reference protein complex, the reference protein complex including two wild-type protein chains;
for every two heavy atoms that belong respectively to the wild-type protein chains of the reference protein complex, calculating an Euclidean distance between the two heavy atoms as an interatomic distance based on the spatial coordinate sets respectively of the two heavy atoms;
identifying, based on the interatomic distances calculated in the step of calculating an Euclidean distance, all interaction interfaces in the reference protein complex, wherein each of the interaction interfaces is between two residues respectively of the wild-type protein chains and wherein a distance between two α-carbons respectively of the residues is less than 5 Å;
selecting one of the interaction interfaces that is related to a specific residue pair, the specific residue pair including a specific residue at a site of interest in one of the wild-type protein chains of the reference protein complex and a paired residue in the other one of the wild-type protein chains of the reference protein complex;
determining, according to information related to properties of side-chain dihedral angles and bond rotation of amino acids, a mutant residue that possibly results from mutation of the specific residue of the reference protein complex and that changes the reference protein complex into a mutant protein complex;
obtaining an inferred rotation angle that is related to a side chain of the specific residue of the reference protein complex from amino acid structure data, the amino acid structure data containing information related to properties of backbone dihedral angles, side-chain dihedral angles and bond rotation of amino acids;
calculating spatial coordinate sets respectively of all heavy atoms of the mutant residue based on the spatial coordinate sets of all heavy atoms of the specific residue of the reference protein complex and the inferred rotation angle;
for a target interface between the mutant residue and a paired residue of the mutant protein complex that respectively correspond to the specific residue and the paired residue of the specific residue pair of the reference protein complex,
for every two heavy atoms respectively of the mutant residue and the paired residue of the mutant protein complex, calculating a value of atomic-level energy and an Euclidean distance based on the spatial coordinate sets of the heavy atoms of the reference protein complex and the spatial coordinate sets of the heavy atoms of the mutant residue of the mutant protein complex, and
calculating, based on the values of atomic-level energy and the Euclidean distances thus calculated, an atomic distance related to the target interface and an atomic interaction force of the target interface;
obtaining relevant information that is related to the specific residue of the reference protein complex and the mutant residue of the mutant protein complex from amino acid physicochemical properties data, the amino acid physicochemical properties data containing information related to physicochemical properties of amino acids; and
estimating binding free energy of the target interface by feeding, into a model for estimating binding free energy, the atomic distance related to the target interface, the atomic interaction force of the target interface and the relevant information, wherein the model for estimating binding free energy is implemented by a deep neural network (DNN).

2. The method as claimed in claim 1, wherein:
the model for estimating binding free energy is trained by using a plurality of training sets that respectively correspond to a plurality of training protein complexes, each of the training protein complexes including at least one pair of training residues that are respectively in two protein chains of the training protein complex and that are related to a training interaction interface; and
each of the training sets contains, for each of the at least one pair of training residues included in the corresponding one of the training protein complexes, an atomic distance that is related to the training interaction interface to which the pair of training residues are related, an atomic interaction of the training interaction interface to which the pair of training residues are related, binding free energy of the training interaction interface to which the pair of training residues are related, and information related to physicochemical properties of amino acids that are related to the pair of training residues.

3. The method as claimed in claim 1, wherein the step of calculating a value of atomic-level energy is to calculate, for each mutant-residue-paired-residue heavy atom pair which includes two heavy atoms respectively of the mutant residue and the paired residue of the mutant protein complex, the value of atomic-level energy as a sum of values of Van der Waals force, hydrogen bond, π-π stacking interaction and electrostatic force between the two heavy atoms of the mutant-residue-paired-residue heavy atom pair.

4. The method as claimed in claim 1, wherein the step of calculating an atomic distance related to the target interface and an atomic interaction force of the target interface includes sub-steps of:
calculating the atomic distance as an average of the Euclidean distances of all mutant-residue-paired-residue heavy atom pairs of the mutant protein complex; and
calculating the atomic interaction force as a sum of the values of atomic-level energy of all mutant-residue-paired-residue heavy atom pairs of the mutant protein complex.

5. A computing system for estimating binding free energy of a mutant protein complex, said computing system comprising:
a storage device configured to store aminoacid structure data, amino acid physicochemical properties data and a model for estimating binding free energy, the amino acid structure data containing information related to properties of backbone dihedral angles, side-chain dihedral angles and bond rotation of amino acids, the amino acid physicochemical properties data containing information related to physicochemical properties of amino acids, the model for estimating binding free energy being implemented by a deep neural network (DNN);
an input module configured to receive protein structure data that contains spatial coordinate sets of all atoms of a reference protein complex, the reference protein complex including two wild-type protein chains;
an output module; and
a processor electrically connected to said storage device, said input module and said output module, and configured to
obtain spatial coordinate sets respectively of all heavy atoms of the reference protein complex from the protein structure data,
for every two heavy atoms that belong respectively to the wild-type protein chains of the reference protein complex, calculate an Euclidean distance between the two heavy atoms as an interatomic distance based on the spatial coordinate sets respectively of the two heavy atoms,
identify, based on the interatomic distances thus calculated, all interaction interfaces in the reference protein complex, wherein each of the interaction interfaces is between two residues respectively of the wild-type protein chains and wherein a distance between two α-carbons respectively of the residues is less than 5 Å,
select one of the interaction interfaces that is related to a specific residue pair, the specific residue pair including a specific residue at a site of interest in one of the wild-type protein chains of the reference protein complex and a paired residue in the other one of the wild-type protein chains of the reference protein complex,
determine, according to information related to properties of side-chain dihedral angles and bond rotation of amino acids, a mutant residue that possibly results from mutation of the specific residue of the reference protein complex and that changes the reference protein complex into a mutant protein complex,
obtain an inferred rotation angle that is related to a side chain of the specific residue of the reference protein complex from the amino acid structure data,
calculate spatial coordinate sets respectively of all heavy atoms of the mutant residue based on the spatial coordinate sets of all heavy atoms of the specific residue of the reference protein complex and the inferred rotation angle,
for a target interface between the mutant residue and a paired residue of the mutant protein complex that respectively correspond to the specific residue and the paired residue of the specific residue pair of the reference protein complex,
for every two heavy atoms respectively of the mutant residue and the paired residue of the mutant protein complex, calculate a value of atomic-level energy and an Euclidean distance based on the spatial coordinate sets of the heavy atoms of the reference protein complex and the spatial coordinate sets of the heavy atoms of the mutant residue of the mutant protein complex, and
calculate, based on the values of atomic-level energy and the Euclidean distances thus calculated, an atomic distance related to the target interface and an atomic interaction force of the target interface,
obtain relevant information that is related to the specific residue of the reference protein complex and the mutant residue of the mutant protein complex from the amino acid physicochemical properties data,
estimate binding free energy of the target interface by feeding, into the model for estimating binding free energy, the atomic distance related to the target interface, the atomic interaction force of the target interface and the relevant information, and
control said output module to present the binding free energy of the target interface thus estimated.

6. The computing system as claimed in claim 5, wherein:
the model for estimating binding free energy is trained by using a plurality of training sets that respectively correspond to a plurality of training protein complexes which are obtained from a protein database, each of the training protein complexes including at least one pair of training residues that are respectively in two protein chains of the training protein complex and that are related to a training interaction interface; and
each of the training sets contains, for each of the at least one pair of training residues included in the corresponding one of the training protein complexes, an atomic distance that is related to the training interaction interface to which the pair of training residues are related, an atomic interaction force of the training interaction interface to which the pair of training residues are related, binding free energy of the training interaction interface to which the pair of training residues are related, and information related to physicochemical properties of amino acids that are related to the pair of training residues.

7. The computing system as claimed in claim 5, wherein the model for estimating binding free energy includes an input layer for receiving the atomic distance, the atomic interaction force and the relevant information, a plurality of hidden layers, and an output layer for outputting the binding free energy thus estimated.

8. The computing system as claimed in claim 5, wherein said processor is further configured to calculate, for each mutant-residue-paired-residue heavy atom pair which includes two heavy atoms respectively of the mutant residue and the paired residue of the mutant protein complex, the value of atomic-level energy as a sum of values of Van der Waals force, hydrogen bond, π-π stacking interaction and electrostatic force between the two heavy atoms of the mutant-residue-paired-residue heavy atom pair.

9. The computing system as claimed in claim 5, wherein said processor is further configured to:
calculate the atomic distance as an average of the Euclidean distances of all mutant-residue-paired-residue heavy atom pairs of the mutant protein complex; and
calculate the atomic interaction force as a sum of the values of atomic-level energy of all mutant-residue-paired-residue heavy atom pairs of the mutant protein complex.
